(19) United States
(12) Patent Application Publication
(10) Pub. No.: US 2025/0339658 A1
(43) Pub. Date: Nov. 6, 2025

(54) AQUEOUS SOLUTION DELIVERY DEVICE

(71) Applicant: Foamtec International Co., Ltd, Waco, TX (US)

(72) Inventors: Thng Cheok Long, Singapore (SG); Michael Edward Strauss, Summerfield, NC (US)

(21) Appl. No.: 19/199,609

(22) Filed: May 6, 2025

Related U.S. Application Data
(60) Provisional application No. 63/643,208, filed on May 6, 2024.

(51) Int. Cl. A61M 35/00

(52) U.S. Cl. CPC A61M 35/006; A61M 2202/04; A61M 2205/0216; A61M 2205/3334

(57) ABSTRACT

An aqueous solution delivery device, especially an aqueous solution delivery device that enables controlled, metered delivery of an aqueous solution to an applicator such as a swab for medical and cleaning applications. The invention includes a swab applicator having a self-contained liquid that includes a main body having a proximal end, a distal end, and a channel located between the proximal end and the distal end where the channel has at least one opening, a bulb containing a liquid secured to the proximal end of the main body, and a swab secured to the distal end of the main body.

### CROSS-REFERENCE TO RELATED APPLICATIONS

This application claims priority to provisional patent application having Ser. No. 63/643,208, filed May 6, 2024, which is herein incorporated by reference in its entirety.

### FIELD OF INVENTION

The present invention generally relates to an aqueous solution delivery device, especially an aqueous solution delivery device that enables the controlled, metered delivery of an aqueous solution to an applicator such as a swab. More particularly, the present invention relates to an aqueous solution delivery device which includes a main body having a closed top, a closed bottom, and a channel located between the closed top and the closed bottom with at least one opening therein positioned near the closed bottom that enables a liquid to escape from the channel to an exterior of the main body; a hollow bulb member having a liquid contained therein secured to the main body such that the closed top of the main body and the liquid are both contained within the hollow bulb member; and a swab member secured to the main body such that the closed bottom and the at least one opening in the channel are contained within the swab member.

### BACKGROUND OF THE INVENTION

Liquids are often supplied to a surface by using a swab applicator that is saturated in a liquid. Saturated swabs are used in medical applications to topically apply antiseptics and medicants. Saturated swabs are also used in cleaning applications to act as carriers for solvents, dissolving agents, or other liquid cleaning agents which then secondarily function to collect debris. Most current swab applicators are either pre-saturated in flexible packaging or are dry applicators to which an aqueous solution is applied at the point-of-use. Both types of swab applicators have disadvantages. Pre-saturated applicators require lengthy and costly stability testing and use excess liquid to ensure efficacy and stability of the liquid to be applied. Dry applicators make exact dosing difficult to perform.

Accordingly, there is a need for a simple, aqueous solution delivery device such as a swab applicator having a self-contained liquid which overcomes these obstacles. There is also a need for a swab applicator having a self-contained liquid which enables the controlled, metered delivery of an aqueous solution in both medical and cleaning applications.

### SUMMARY OF THE INVENTION

The present invention is directed to an aqueous solution delivery device and, more particularly, to a swab applicator having a self-contained liquid which, in one exemplary embodiment, includes a main body having a closed top, a closed bottom, and a channel located between the closed top and the closed bottom having at least one opening therein positioned near the closed bottom that enables a liquid to escape from the channel to an exterior of the main body, a hollow bulb member having a liquid contained therein secured to the main body such that the closed top of the main body and the liquid are both contained within the hollow bulb member, and a swab member secured to the main body such that the closed bottom and the at least one opening in the channel are contained within the swab member. In another exemplary embodiment, the main body preferably includes a weakened area surrounding the channel that can be broken by applying pressure thereto to enable the liquid contained in the hollow bulb member to enter the channel and contact the swab through the at least one opening contained in the channel. This weakened area surrounding the channel of the main body may be positioned on the main body at a location where the liquid contained within the hollow bulb member surrounds the main body and its channel and where the liquid is at or near the bottom of the hollow bulb member near where the hollow bulb member is secured to the main body. The weakened area surrounding the channel of the main body may be marked by a score line which identifies the area where a user can apply pressure to break the main body thereby enabling liquid contained within the hollow bulb member to enter the channel of the main body. Once the liquid enters the channel of the main body it can be transferred from the hollow bulb member to the at least one opening in the channel located near the closed end of the main body and into the swab member.

The at least one opening in the channel may comprise a plurality of openings and the plurality of openings may be spaced equidistant from one another. The swab member may comprise a foam swab and the foam swab may be comprised of polyurethane. The main body of the aqueous solution delivery device may comprise a single channel, such as a tubular enclosed passage, having a closed top and a closed bottom, and at least one opening positioned near the closed bottom, along with a weakened area of the tubular enclosed passage that can be broken to expose the interior of the tubular enclosed passage by applying pressure to the weakened area. This allows a liquid to enter the interior of the tubular enclosed passage. The weakened area of the main body may be marked with a score line to enable a user of the device to easily identify the weakened area where pressure may be applied to break the tubular enclosed passage to allow a liquid to enter the interior of the tubular enclosed passage. The main body of the aqueous delivery system may also include a reinforced portion or area located beneath the weakened area of the of the tubular enclosed passage that is stronger than the main body located above the weakened area so that the tubular enclosed passage of the bottom of the main body remains intact for a liquid to enter the interior of the tubular enclosed passage once the weakened area is broken. In addition, an area of the main body located within the hollow bulb member and above the score line and/or the weakened area of the tubular enclosed passage may be slightly reinforced so that it forms a break-off stem for a user. The break off stem may be tubular or solid. When the user applies force to the break-off stem contained within the hollow bulb member, the weakened area of the main body should easily break thereby providing access to the interior of the channel of the main body. The parts of the main body comprising the closed top, the break-off stem, the weakened area, the score line identifying the weakened area, the reinforced area located below the weakened area, the channel having at least one opening therein, and the closed bottom may all be formed from one molded piece.

The main body of the aqueous solution delivery system may be comprised of one or more polymers and may be transparent so that a user can see a liquid contained within the hollow bulb member travel into the channel of the main body member after the weakened area of the body member is broken. The hollow bulb member of the aqueous solution delivery device may be semi-flexible and may be comprised of one or more elastomers. The hollow bulb member may also be transparent so that a user can see a liquid contained within the hollow bulb member travel from the hollow bulb member into the channel of the main body once the weakened area of the main body is broken. The bulb member and/or the swab member may be permanently secured to the main body.

In yet another exemplary embodiment, the present invention is directed to a swab applicator having a self-contained liquid that includes a main body having a proximal end, a distal end, a channel located between the proximal end and the distal end, and at least one opening in the channel located near the distal end of the main body, a bulb having a liquid chamber containing a liquid secured to the main body such that the proximal end of the main body is contained within the bulb, and a swab member secured to the main body such that the distal end of the main body is contained within the swab member. The at least one opening in the channel located near the distal end of the main body may be located at the distal end of the main body such that a liquid traveling through the channel exits the distal end of the main body and disperses into the swab member. Alternatively, the at least one opening in the channel located near the distal end of the main body may include a plurality of openings that are located near the distal end of the main body where the distal end of the main body is closed so that a liquid traveling through the channel to the distal end of the main body can be dispersed through the plurality of openings in the channel and into the swab member. The plurality of openings in the channel may be spaced equidistantly from one another so that a liquid traveling through the channel and into the swab member is evenly dispersed throughout the swab member.

In still another exemplary embodiment, the main body of the swab applicator having a self-contained liquid may include a weakened area about the channel that can be broken by applying pressure to it to enable a liquid contained within the liquid chamber of the bulb to enter the channel and contact the swab member through at least one opening contained in the channel. The weakened area about the channel may be marked with a score line to indicate where pressure should be applied to break the main body to enable liquid in the bulb to enter the channel of the main body. The parts of the main body comprising the proximal end, the distal end, the channel having at least one opening, the weakened area, and the score line identifying the weakened area may all be formed from one molded piece. The main body and/or the bulb may be transparent. The swab member may be a foam swab member that may be comprised of polyurethane. The bulb member and/or the swab member may be permanently secured to the main body.

### BRIEF DESCRIPTION OF THE DRAWINGS

The subject invention will hereinafter be described in conjunction with the appended drawing figures, wherein like numerals demote like elements, and

FIG. 1 is side cross-sectional view of one exemplary embodiment of the aqueous solution deliver device of the present invention;

FIG. 2 is a side cross-sectional view of another exemplary embodiment of the aqueous solution delivery device of the present invention having 0.5 mL of liquid contained within a bulb member for delivery to the swab member;

FIG. 3 is a side cross-sectional view of the exemplary embodiment of the aqueous solution delivery device shown in FIG. 2 having 1.0 mL of liquid contained within the bulb member for delivery to the swab member; and

FIG. 4 is a side cross-sectional view of the exemplary embodiment of the aqueous solution delivery device shown in FIGS. 2 and 3 having 1.5 mL of liquid contained within the bulb member for delivery to the swab member along with possible length, width, and diameter measurements for the device and some of its components.

### DETAILED DESCRIPTION OF EXEMPLARY EMBODIMENTS

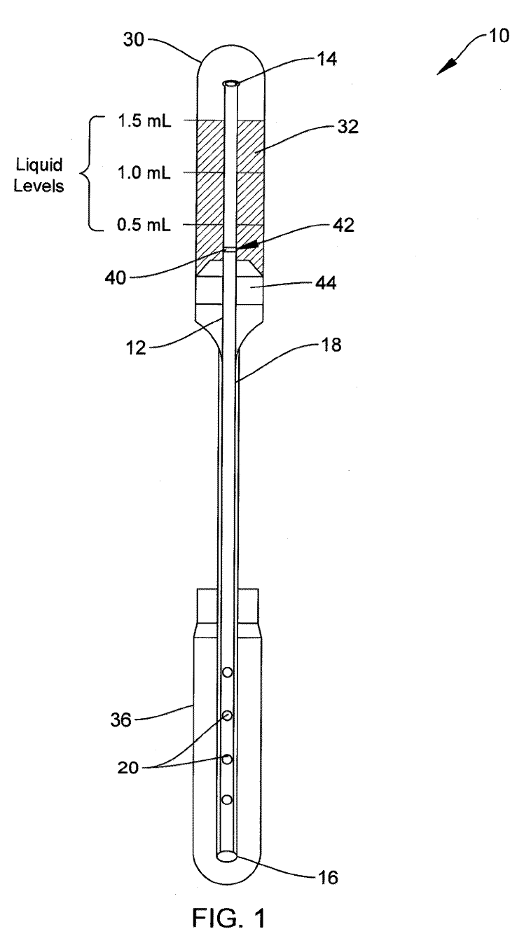

The aqueous solution delivery device 10 of the present invention generally provides a swab applicator having a self-contained liquid. In one exemplary embodiment shown in FIG. 1, the aqueous solution delivery system 10 includes a main body 12 having a closed top 14, a closed bottom 16, a channel 18 located between the closed top 14 and the closed bottom 16 having at least one opening 20 positioned near the closed bottom 16 that enables a liquid to escape from the channel 18 to an exterior of the main body 12, a hollow bulb member 30 having a liquid 32 contained therein secured to the main body 12 such that the closed top 14 of the main body 12 and the liquid 32 are both contained within the hollow bulb member 30, and a swab member 36 secured to the main body 12 such that the closed bottom 16 and the at least open opening 20 in the channel 18 are contained within the swab member 36.

The at least one opening 20 positioned near the closed bottom 16 of the main body 12 may comprise a disbursement array having a plurality of openings that are located equidistant from one another. The main body 12 preferably includes a weakened area 40 surrounding the channel 18 that can be broken by applying pressure thereto to enable the liquid 32 contained in the hollow bulb member 30 to enter the channel 18 and contact the swab member 36 through the at least one opening 20 contained in the channel 18. In another exemplary embodiment of the aqueous solution delivery system, the weakened area 40 surrounding the channel is marked with a score line 42 to indicate where pressure should be applied to break the main body 12 to enable liquid 32 in the hollow bulb member 30 to enter the channel 18 of the main body 12. The main body 12 of the aqueous delivery system 10 may also include a reinforced portion or area 44 located beneath the weakened area 40 of the of the main body 12 that is stronger than the main body 12 located above the weakened area 40 so that the tubular enclosed passage of the bottom portion of the main body 12 remains intact for a liquid 32 to enter the channel 18 of the main body 12 once the weakened area 40 is broken. In addition, an area of the main body 12 located within the hollow bulb member 30 and above the score line 42 and/or the weakened area 40 of the main body 12 may be slightly reinforced so that it forms a break-off stem 46 for a user. The break off stem may be tubular or solid. When the user applies force to the break-off stem 46 contained within the hollow bulb member 30, the weakened area 40 of the main body 12 should easily break thereby providing access to the interior of the channel 18 of the main body 12. The parts of the main body 12 comprising the closed top 14, the break-off stem 46, the weakened area 40, the score line 42 identifying the weakened area 40, the reinforced area 44 located below the weakened area 40, the channel 18 having at least one opening 20 therein, and the closed bottom 16 may all be formed from one molded piece.

The hollow bulb member 30 of the aqueous solution delivery device 10 may be semi-flexible and may be comprised of one or more elastomers. The main body 12 of the aqueous solution deliver system 10 may be comprised of one or more polymers and may be transparent so that a user can see a liquid contained within the hollow bulb member 30 travel into the channel 18 of the main body member 12 after the weakened area 40 of the body member 12 is broken by applying pressure to the break off stem 46 contained within the hollow bulb member 30. The hollow bulb member 30 may also be transparent and the hollow bulb member 30 and/or the swab member 36 may be permanently secured to the main body 12.

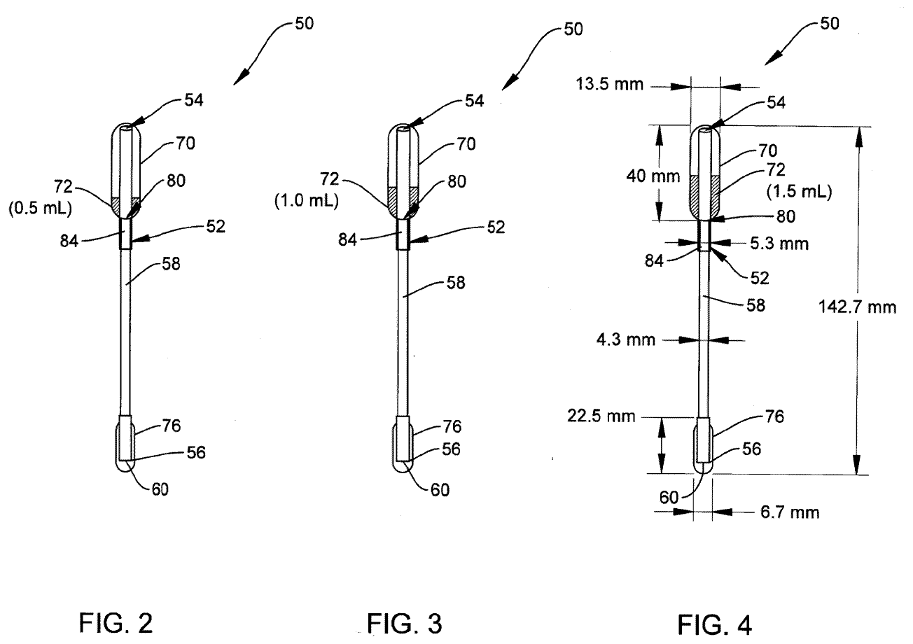

Once the weakened area 40 of the main body 12 is broken and the liquid 32 enters the channel 18, the liquid 32 travels down the channel 18 to the at least one opening 20 or disbursement array contained in the channel 18 which then enables the liquid 32 to exit the channel 18 and enter the swab member 36 in metered amounts. The hollow bulb member 30 may include various levels of liquid or aqueous solution as indicated by liquid levels marked as 1.5 mL, 1.0 mL, and.5 mL in FIG. 1 and as shown in FIGS. 2-4. The hollow bulb member 30 allows for controlled amounts of the liquid 32 or aqueous solution to be applied to the swab member 36. The aqueous solution delivery system of the present invention is a single, self-contained unit designed for one-time use.

The controlled, metered delivery of the liquid/aqueous solution 32 in the hollow bulb member 30 to the swab member 36 enables a user to saturate the swab member 36 with an exact amount of the liquid/aqueous solution 32 that is needed for a particular application. Since there is no time for any active ingredients contained in the liquid 32 to bind to the swab member 36 prior to application, no excess liquid 32 is needed thereby resulting in cost savings. In addition, since the liquid/aqueous solution 32 stays separate from the swab member 36 until the time of application, the strength of the liquid/aqueous solution 32 remains consistent prior to application thereby extending its shelf life as opposed to when it is used in pre-saturated swabs. As a result, the expiration date and efficacy of the liquid/aqueous solution will rely solely on the liquid/aqueous solution and not the combination of the liquid/aqueous solution and the applicator (swab) material.

FIGS. 2-4 show cross-sectional views of another exemplary embodiment of the invention directed to a swab applicator having a self-contained liquid. FIGS. 2-4 show the same exemplary embodiment of the invention containing different amounts of liquids-namely 0.5 mL, 1.0 mL, and 1.5 mL of liquid. The liquid may be any type of liquid or aqueous solution depending upon the desired application for the swab member once saturated. For example, the liquid may comprise iodine for medical applications or an alcohol based solution for cleaning applications. The exemplary embodiment of the applicator device 50 shown in FIGS. 2-4 may include a main body 52 having a proximal end 54, a distal end 56, a channel 58 located between the proximal end 54 and the distal end 56, and at least one opening 60 in the channel 58 located near the distal end 58 of the main body 52, a bulb 70 having a liquid chamber containing a liquid 72 secured to the main body 52 such that the proximal end 54 of the main body 52 is contained within the bulb 70, and a swab member 76 secured to the main body 52 such that the distal end 56 of the main body 52 is contained within the swab member 76. The at least one opening 60 in the channel located near the distal end 56 of the main body 52 may be located at the distal end 56 of the main body 52 such that a liquid 72 traveling through the channel 58 exits the distal end 56 of the main body 52 and disperses into the swab member 76. Alternatively, the at least one opening in the channel located near the distal end of the main body may include a plurality of openings that are located near the distal end of the main body where the distal end of the main body is closed so that a liquid traveling through the channel to the distal end of the main body can be dispersed through the plurality of openings in the channel and into the swab member. The plurality of openings in the channel may be spaced equidistantly from one another so that a liquid traveling through the channel and into the swab member is evenly dispersed throughout the swab member.

Main body 52 may also include a weakened and/or scored area 80 about the channel 58 that can be broken by applying pressure to it to enable a liquid 72 contained within the liquid chamber of the bulb 70 to enter the channel 58 and contact the swab member 76 through at least one opening 60 contained in the channel 58. The weakened and/or scored area 80 about the channel 58 indicates where pressure should be applied to break the main body 52 to enable liquid 72 in the bulb 70 to enter the channel 58 of the main body 52. Main body 52 may also include a reinforced area 84 located beneath the weakened and/or scored area 80 so that the channel 58 comprising the lower part of the main body 52 remains intact for a liquid 72 to enter the channel 58 of the main body 52 once the weakened and/or scored area 80 is broken. The parts of the main body comprising the proximal end 54, the distal end 56, the channel 58 having at least one opening 60, the weakened and/or scored area 80, and the reinforced area 84 may all be formed from one molded piece. The main body and/or the bulb may be transparent. The swab member may be a foam swab member that may be comprised of polyurethane. The bulb member and/or the swab member may be permanently secured to the main body.

Although FIGS. 2-4 have bulb members 70 that contain 0,5 mL, 1.0 mL, and 1.5 mL of liquid, respectively, it is possible for applicator device 50 to have 1.5 mL in bulb member 70 that can be dispensed in metered amounts by having multiple different weakened and/or scored areas 80 as part of the main body 12 so that applying pressure to one weakened and/or scored area 80 would only allow an amount of liquid 72 contained above that weakened and/or scored area 80 to enter channel 58 of main body 52 once the weakened and/or scored area 80 is broken.

In one exemplary embodiment, bulb 70 may have a width of 13.5 millimeters and a length of 40 millimeters, reinforced area 84 may have a width of 5.3 millimeters, channel 58 may have a width of 4.3 millimeters, swab member 76 may have a width of 6.7 millimeters and a length of 22.5 millimeters, and the entire device 50 may have a length of 142.7 millimeters. However, it should be appreciated that various length, width and diameter measurements of the various components of the exemplary embodiments of the device of the present invention will change depending on the intended application or applications for the device.

The drawings and description of exemplary embodiments of the invention herein shows various exemplary embodiments of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included examples are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning.

1. An aqueous solution delivery device comprising:
a main body having a closed top, a closed bottom, and a channel located between the closed top and the closed bottom wherein the channel includes at least one opening therein positioned near the closed bottom that enables a liquid to escape from the channel to an exterior of the main body;
a hollow bulb member having a liquid contained therein secured to the main body such that the closed top of the main body and the liquid are contained within the hollow bulb member; and
a swab member secured to the main body such that the closed bottom and the at least one opening in the channel are contained within the swab member.

2. The aqueous solution delivery device of claim 1 wherein the main body includes a weakened surrounding the channel that can be broken by applying pressure thereto to enable the liquid contained in the hollow bulb member to enter the channel and contact the swab member through the at least one opening contained in the channel.

3. The aqueous solution delivery system of claim 2 wherein the weakened area surrounding the channel is marked with a score line.

4. The aqueous solution delivery device of claim 1 wherein the at least one opening in the channel comprises a plurality of openings that are spaced equidistantly from one another.

5. The aqueous solution delivery system of claim 1 wherein the swab member is foam swab.

6. The aqueous solution delivery system of claim 5 wherein the foam swab is comprised of polyurethane.

7. The aqueous solution delivery system of claim 1 wherein the main body is comprised of one or more polymers.

8. The aqueous solution delivery system of claim 1 wherein the main body is transparent.

9. The aqueous solution delivery system of claim 1 wherein the hollow bulb member is semi-flexible.

10. The aqueous solution delivery system of claim 9 wherein the semi-flexible hollow bulb member is comprised of one or more elastomers.

11. The aqueous solution delivery system of claim 1 wherein the hollow bulb member is transparent.

12. The aqueous solution delivery system of claim 1 wherein the hollow bulb member is permanently secured to the main body.

13. The aqueous solution delivery system of claim 1 wherein the swab member is permanently secured to the main body.

14. The aqueous solution delivery system of claim 2 wherein the main body includes a reinforced portion surrounding the channel located beneath the weakened area of the main body that is stronger than the main body located above the weakened area of the main body.

15. The aqueous solution delivery system of claim 3 wherein the main body includes a reinforced portion surrounding the channel located beneath the score line on the main body that is stronger than the main body located above the score line.

16. A swab applicator having a self-contained liquid comprising:
a main body having a proximal end, a distal end, a channel located between the proximal end and the distal end, and at least one opening in the channel located near the distal end of the main body;
a bulb having a liquid chamber containing a liquid secured to the main body such that the proximal end of the main body is contained within the bulb; and
a swab member secured to the main body such that the distal end of the main body is contained within the swab member.

17. The swab applicator having a self-contained liquid of claim 16 wherein the main body includes a weakened area about the channel that can be broken by applying pressure thereto to enable the liquid contained in the liquid chamber of the bulb to enter the channel and contact the swab member through the at least one opening contained in the channel.

18. The swab applicator having a self-contained liquid of claim 17 wherein the at least one opening in the channel located near the distal end of the main body is located at the distal end of the main body.

19. The swab applicator having a self-contained liquid of claim 17 wherein the proximal end of the main body is closed.

20. The swab applicator having a self-contained liquid of claim 17 wherein the weakened area about the channel is marked with a score line to indicate where pressure should be applied to break the main body to enable the liquid in the bulb to enter the channel of the main body.
